(19) United States
(12) Patent Application Publication
(10) Pub. No.: US 2008/0257412 A1
(43) Pub. Date: Oct. 23, 2008

(54) Fluid Delivery Device With Flow Rate Control

(76) Inventor: John Howard Gordon, Salt Lake City, UT (US)

Correspondence Address: Microlin, LLC, 2425 South 900 West, Salt Lake City, UT, 84119, US

(21) Appl. No.: 12/106,205

(22) Filed: Apr. 18, 2008

Related U.S. Application Data
(60) Provisional application No. 60/912,463, filed on Apr. 18, 2007.

(51) Int. Cl. F17D 3/18; F17D 3/01; F17D 1/00; A61M 31/00; A61M 1/00

(52) U.S. Cl. 137/8; 137/110; 604/500; 604/67

(57) ABSTRACT

A device for delivering a fluid to a target site is disclosed in one embodiment of the invention as including a pump, a flow modulator, a flow meter, and a controller. The pump may generate a fluid stream characterized by a flow rate. The flow modulator may smooth out irregularities in the flow rate, thereby generating a second fluid stream having a second flow rate that is substantially more uniform than the first flow rate. The flow meter may measure the flow rate of the second fluid stream. The controller may then compare the flow rate of the second fluid stream to a target flow rate, and adjust the pump speed to substantially align the second flow rate with the target flow rate.

### CROSS-REFERENCE TO RELATED APPLICATION

This application claims the benefit of U.S. Provisional Patent Application No. 60/912,463 filed Apr. 18, 2007, which application is incorporated by reference.

### FIELD OF THE INVENTION

This invention relates to fluid delivery systems, and more particularly to systems and methods for delivering a fluid to a target location at a substantially uniform delivery rate.

### BACKGROUND

Escalating costs of health care and advances in laparoscopic and other surgical techniques have encouraged minimally invasive surgical procedures with short associated recovery times. To facilitate early patient discharge while providing adequate post-operative pain management, portable drug delivery systems for in-home use are commonly prescribed following surgery.

A portable drug delivery system may continuously infuse pain-relieving drugs into the patient at a predetermined basal rate, usually ranging between about three and about ten milliliters per hour, to provide regional pain management. In some applications, the patient may press a bolus button on a controller to temporarily increase the rate for a preset period of time. Drug delivery may thus be customized according to the patient's perception of pain, while simultaneously preventing the patient from exceeding a safe, prescribed drug dosage.

Various electromechanical pumps have been developed for use in connection with portable drug delivery systems. One type of delivery system, for example, uses a driving force to pump fluid through a length of small diameter tubing where the diameter of the tubing controls the flow rate of the fluid. This type of delivery system, while inexpensive to manufacture and implement, is inherently inaccurate. Particularly, it is subject to delivery rate fluctuations caused by variations in liquid viscosity due to temperature, variations in head backpressure due to height differences between a drug reservoir and the drug infusion site, and variations in fill level of the elastomeric pump.

Peristaltic pumps, on the other hand, rely on controlled rotor motion to provide precisely adjustable delivery rates. Such pumps, however, tend to be less portable and more expensive than other types of pumps. Moreover, because delivery rate is calculated indirectly according to the revolution rate of the rotor, inaccuracies may occur.

Conventional diaphragm-type pumps, piezo pumps, or elastomeric balloon-type pumps, may provide a low-cost alternative for providing effective drug delivery, however, delivery rates effectuated by such pumps are subject to fluctuations caused by variations in liquid viscosity due to temperature, as well as variations in head backpressure. Reliable methods for measuring delivery rates are thus critical to enabling adjustments in diaphragm stroke amplitude and frequency as needed to achieve and maintain a desired rate of delivery.

In view of the foregoing, what is needed is a device and method for delivering fluids to a target site at a substantially uniform delivery rate. Ideally, such a device and method would enable use of non-continuous, diaphragm-type and piezo pumps, facilitate accurate delivery rate measurements, and minimize power supply requirements. Such a device and method is disclosed and claimed herein.

### SUMMARY OF THE INVENTION

The invention has been developed in response to the present state of the art and, in particular, in response to the problems and needs in the art that have not yet been fully solved by currently available fluid delivery systems. Accordingly, the invention has been developed to provide novel devices and methods for delivering fluids to a target location at an adjustable, uniform delivery rate. The features and advantages of the invention will become more fully apparent from the following description and appended claims and their equivalents, and also any subsequent claims or amendments presented, or may be learned by practice of the invention as set forth hereinafter.

Consistent with the foregoing, a device for delivering a fluid to a target site may include a pump, a flow modulator, a flow meter, and a controller. The pump may generate a fluid stream characterized by a flow rate. The flow rate may depend on a speed at which the pump is operated. In some embodiments, the pump may operate intermittently such that the flow rate is substantially pulsatile. The pump may include, for example, a piezo pump, a diaphragm-type pump, a centrifugal pump, a peristaltic pump, or a piston-type pump.

The flow modulator may smooth out irregularities in the flow rate, thereby generating a second fluid stream having a second flow rate that is substantially more uniform than the first flow rate. In certain embodiments, the flow modulator may include a volume expansion zone to expand upon receiving the first fluid stream. The volume expansion zone may then contract to generate the second fluid stream.

The flow meter may measure the flow rate of the second fluid stream. The controller may receive the flow rate measurement from the flow meter and compare the flow rate of the second fluid stream to a target flow rate. The controller may then adjust the pump speed to substantially align the second flow rate with the target flow rate.

In some embodiments, the device may further include a fluid reservoir to retain the fluid prior to delivery. The device may also include a power source for supplying power to the controller, a check valve to prevent fluid backflow from the flow modulator to the pump, and/or a flow restrictor to regulate the first or second fluid stream. In one embodiment, the device further includes a bypass valve to enable the first or second fluid stream to bypass a flow restrictor.

A method to deliver a fluid to a target site at a substantially uniform delivery rate is also presented herein. The method may include generating a fluid stream characterized by a flow rate. In some embodiments, the flow rate may be substantially pulsatile. As used herein, pulsatile may include any flow rate that rises and falls regularly or irregularly, including those generated by a piston pump, piezo pump, diaphragm pump, other types of pumps, pump duty cycles, and the like. Irregularities or variations in the flow rate may be smoothed to generate a second fluid stream having a second, substantially more uniform flow rate. This may be accomplished by introducing the first fluid into a flow modulator, permitting a second fluid stream having a second flow rate that is substantially more uniform than the first flow rate to exit the flow modulator. Introducing the first fluid stream into a flow modulator may comprise receiving the first fluid stream into a volume expansion zone and may further comprise expanding a volume in the volume expansion zone to accumulate fluid within the flow modulator when the first flow rate is greater than the second flow rate. The method may also comprise contracting a volume in the volume expansion zone to expel fluid from the flow modulator when the first flow rate is less than the second flow rate.

The second flow rate may be measured and compared to a target flow rate. The first flow rate may then be adjusted to substantially align the second flow rate with the target flow rate.

In one embodiment, the first fluid stream may be received into a volume expansion zone to facilitate smoothing irregularities in the first fluid rate. The volume expansion zone may be contracted to generate the second fluid stream. In one embodiment, a flow modulator comprises a volume expansion zone that receives a first fluid stream having a first fluid rate. A second fluid stream having a second fluid rate may exit the volume expansion zone of the flow modulator. When the first fluid rate is greater than the second fluid rate, a volume of the volume expansion zone expands allowing the flow modulator to accumulate fluid. When the first flow rate is less than the second flow rate, the volume of the volume expansion zone contracts expelling the fluid from the volume expansion zone and flow modulator. Thus, the second fluid stream that exits the volume expansion zone of the flow modulator has a generally more uniform flow rate. In other words, the irregularities or variations in the flow rate, whether or not regular or purposeful, are “smoothed out” as the first fluid stream transitions into the second fluid stream in the flow modulator.

In some embodiments, the method may further include preventing the first fluid stream from substantially reversing a direction of flow. In other embodiments, a flow restrictor may be provided to regulate a flow of the first or second fluid stream. A bypass valve may be provided to enable the first or second fluid stream to bypass a flow restrictor.

In accordance with another embodiment of the present invention, a device to deliver fluid to a target site at a substantially uniform delivery rate may include means for generating a first fluid stream having a first flow rate. The device may further include means for smoothing out irregularities in the first flow rate to thereby generate a second fluid stream. The second fluid stream may have a second flow rate that is substantially more uniform than the first flow rate.

The device may further include means for measuring the second flow rate, means for comparing the second flow rate to a target flow rate, and means for substantially aligning the second flow rate with the target flow rate. In certain embodiments, the device may also include means for restricting a flow of the first or second fluid stream, and enabling the first or second fluid stream to bypass a means for restricting the flow.

The present invention provides improved devices and methods for delivering a fluid to a target site at a substantially uniform delivery rate. The features and advantages of the present invention will become more fully apparent from the following description and appended claims, or may be learned by practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 is a high-level view of a fluid delivery device shown in relation to a user thereof;

FIG. 2 is a high-level block diagram of one embodiment of a fluid delivery device in accordance with the invention;

FIG. 3 is a high-level block diagram of another embodiment of a fluid delivery device in accordance with the invention;

FIG. 4 is a high-level block diagram of yet another embodiment of a fluid delivery device in accordance with the invention;

FIG. 5A is a high-level diagram of one embodiment of a flow modulator in accordance with the invention;

FIG. 5B is a high-level diagram of another embodiment of a flow modulator in accordance with the invention;

FIG. 6 is a graph showing the flow rate of a fluid delivery device without a flow modulator in accordance with the invention;

FIG. 7 is a graph showing the flow rate of a fluid delivery device with a flow modulator in accordance with the invention; and

FIG. 8 is a high-level diagram of one embodiment of a flow meter for use with the present invention.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The presently described embodiments will be best understood by reference to the drawings, wherein like parts are designated by like numerals throughout.

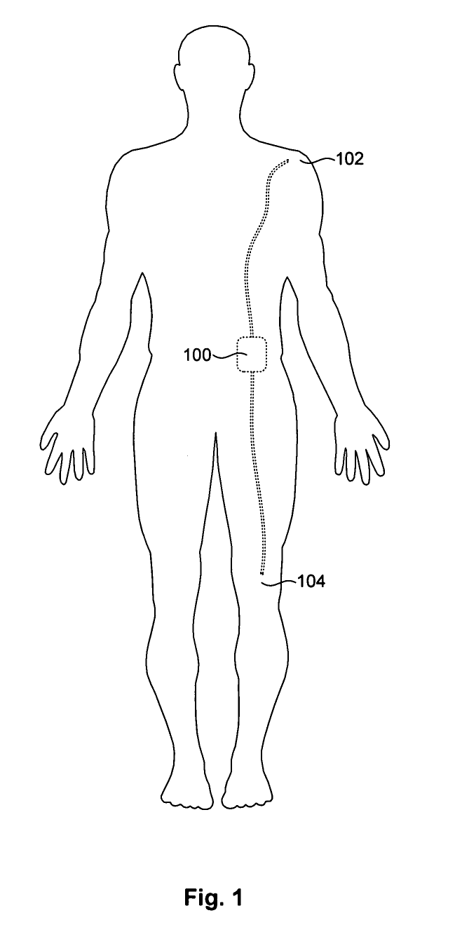

Referring to FIG. 1, in selected embodiments, a fluid delivery device 100 for providing an adjustable, substantially uniform delivery rate may used to deliver a medicine, drug, or some other beneficial agent to the body. One contemplated application for the fluid delivery device 100 is that of regional pain management, although the claimed invention is not limited to this application. For example, a fluid delivery device 100 in accordance with the invention may be configured to deliver pain medication to different areas of the body in order to provide localized pain relief at or near the delivery site or downstream from the delivery site.

Where surgery is performed on a shoulder, knee, or foot, for example, the pain medication may be injected at or near the area of the surgery or upstream from the area of the surgery. The amount of pain medication required for each surgery may vary based on the type of surgery or the area where the medication is delivered. As an example, a first delivery site may require a basal delivery rate of three milliliters per hour whereas a second delivery site may require a basal delivery rate of five milliliters per hour. Thus, the delivery rate of the fluid delivery device 100 may be adjustable to provide a desired level of pain management.

The fluid delivery device 100 may be capable of generating a substantially consistent delivery rate in spite of environmental factors, such as varying head pressure or temperature, which may otherwise affect the delivery rate. For example, the head pressure at or near a shoulder area 102 may differ significantly from the head pressure at or near a knee area 104, particularly where the fluid delivery device 100 is located at or near the patent's beltline. As will be explained in more detail hereafter, because the fluid delivery device 100 includes a flow meter, the fluid delivery device 100 may achieve a substantially consistent delivery rate regardless of the height or location of the target delivery area.

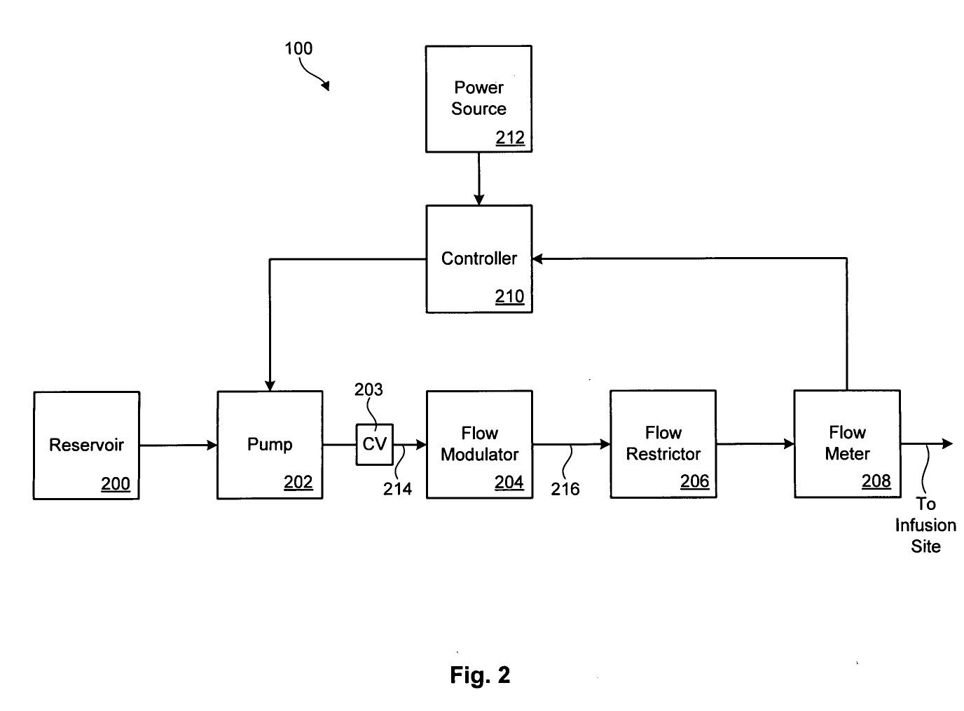

Referring to FIG. 2, in selected embodiments, a fluid delivery device 100 in accordance with the invention may include a fluid-containing reservoir 200, a pump 202, a check valve 203, a flow modulator 204, a flow restrictor 206, a flow meter 208, a controller 210, and a power source 212. A reservoir 200, such as a bag, compartment, syringe, or the like, may contain medications, drugs, or other beneficial agents in liquid form. The pump 202 may draw the liquids out of the reservoir 200 to generate a first fluid stream 214, having a flow rate. The flow rate of the first fluid stream 214 may depend at least partially on the pump speed.

In selected embodiments, a flow modulator 204 may be provided to smooth out irregularities in the flow rate of the first fluid stream 214, thereby generating a second fluid stream 216 with a substantially more uniform flow rate. In selected embodiments, a check valve 203 and flow restrictor 206 may control the flow of liquid into and out of the flow modulator 204. In particular, the check valve 203 may prevent liquid from flowing back toward the pump and the flow restrictor 206 may restrict the flow of liquids out of the flow modulator 204. The flow restrictor 206 may be as simple as an orifice having a selected diameter, tubing with a selected inside diameter, or the like. The importance and role of these components 203, 206 will be explained in more detail hereafter.

After liquid passes through the flow restrictor 206, a flow meter 208 may measure the flow rate of the liquid. This measurement may be received by a controller 210, where it may be compared to a target delivery rate. If the flow rate differs from the target delivery rate, the controller 210 may attempt to align the flow rate with the target delivery rate by adjusting the pump speed. For the purposes of this description, “pump speed” may include parameters such as pump RPM, duty cycle, stroke amplitude, stroke frequency, or the like, depending on the type of pump that is used. A power source 212, such as a battery 212, may provide power to the controller 210, the pump 202, the flow meter 208, and/or other components, if required.

The flow modulator 204 may allow various types of pumps to be run in a non-continuous mode to enable a large turn down ratio (ratio between the highest and lowest flow rate). This enables utilization of larger more efficient pumps of various kinds, including piezo, diaphragm, centrifugal, peristaltic, and piston-type pumps capable of flow rates higher than those typically required. These pumps can be run at their most efficient rate for a period of time, then be shut down for a period of time such that the average pump rate meets a target delivery rate. Using a more efficient, higher capacity pump and running with a duty cycle provides two benefits: first, it provides extra capacity for a bolus above a basal rate; and second, it allows the pump to be run in a more efficient manner such that it makes better use of batteries.

Although operating with a duty cycle may provide various advantages, operating in this manner generates an irregular (e.g., pulsatile) flow rate which may be unsuitable for medication delivery. An irregular flow rate is also difficult to accurately measure with various types of flow meters 208. The flow modulator 204 may smooth out these irregularities to provide a more uniform flow rate. This, in turn, provides a more uniform delivery rate and enables more accurate measurements by the flow meter 208.

One benefit of the flow modulator 204 is that it enables use of lower cost piezo pumps. In general, flow rates generated by a piezo pump are a function of diaphragm stroke amplitude and frequency, two parameters which may be adjusted by the controller 210. The flow rate may also depend on factors such as liquid viscosity, which is highly temperature dependent, and head pressure, which depends on the height differential between the reservoir 200 and the infusion site. By using a flow meter 208, the flow rate may be accurately measured and provide assurance that a particular delivery rate has been achieved and maintained.

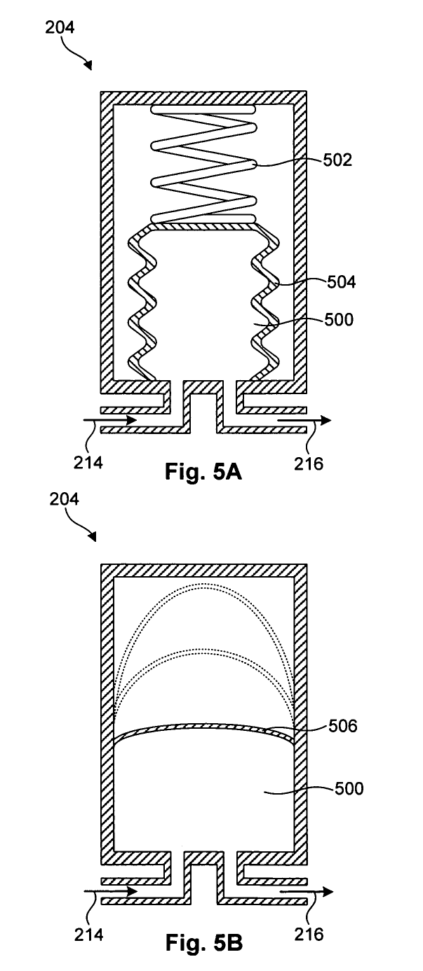

In general, a flow modulator 204 in accordance with the invention may include four features to provide the benefits discussed herein. These features may include (1) a volume expansion zone within the fluid path that can undergo volume changes, (2) means to prevent backflow (e.g., a one-way check valve 203) at the entrance of the volume expansion zone or to the pump, (3) means to create backpressure (e.g., the flow restrictor 206) downstream from the volume expansion zone, and (4) means to act on the boundaries of the volume expansion zone to compress the liquid therein. Several of these features will be discussed in more detail in association with FIGS. 5A and 5B.

In one embodiment, the means to prevent backflow may include the structures discussed herein, and may include without limitation, check valves, passive valves, active valves, other types of valves, flow restrictors, gates, traps, weirs, other ways known to those of skill in the art, and combinations thereof. The means to create backpressure to facilitate accumulation of fluid in the volume expansion zone with the flow modulator 204 may include without limitation the structures discussed herein and may include without limitation, flow restrictors, tube or passage diameters or restrictions, tortuous paths, orifices, control or other types of valves, venturis, or other ways known in the art, and combinations thereof. The means to act on the boundaries of the volume expansion zone, or in other words to contract a volume of the volume expansion zone to expel fluid from the volume expansion zone and consequently from the flow modulator 204, may include the structures discussed herein and may also include without limitation, bellows, biasing members, diaphragms, pistons, valves, balloons and other elastomeric devices, and other ways known in the art to expand and contract a volume, and combinations thereof.

In certain embodiments, the controller 210 may include an interface to allow a user or medical professional to select a basal delivery rate. Such a basal delivery rate, for example, may be selected based on the size of the patient, the amount of pain experienced by the patient, the area of the body treated, or the like. In certain embodiments, the controller interface may also enable a user to select a bolus delivery rate when breakthrough pain or other situations occur which may require a temporary increase in the delivery rate. In certain embodiments, the controller 210 may limit the amount of time the device 100 operates at the bolus delivery rate to prevent harm, abuse, or the like. In certain embodiments, the controller 210 may also limit the frequency for selecting a bolus. For example, once the bolus has been selected and delivered, the controller 210 may require that a user wait several hours before a bolus can be selected again. In other embodiments, the controller 210 may limit the number of times a bolus infusion may be selected in a particular period (e.g., a twenty-four hour period).

The fluid delivery device 100 illustrated in FIG. 2 is provided by way of example and is not intended to be limiting. Various components may be re-arranged, or possibly added or removed without altering the function or principle of operation of the device 100. For example, in certain embodiments, the flow restrictor 206 may be placed downstream from the flow meter 208 or upstream from the flow modulator 204. In other embodiments, additional check valves 203 may be placed at different locations in the fluid stream to prevent backflow. Thus, variations of the device 100 are possible and fall within the scope of the invention.

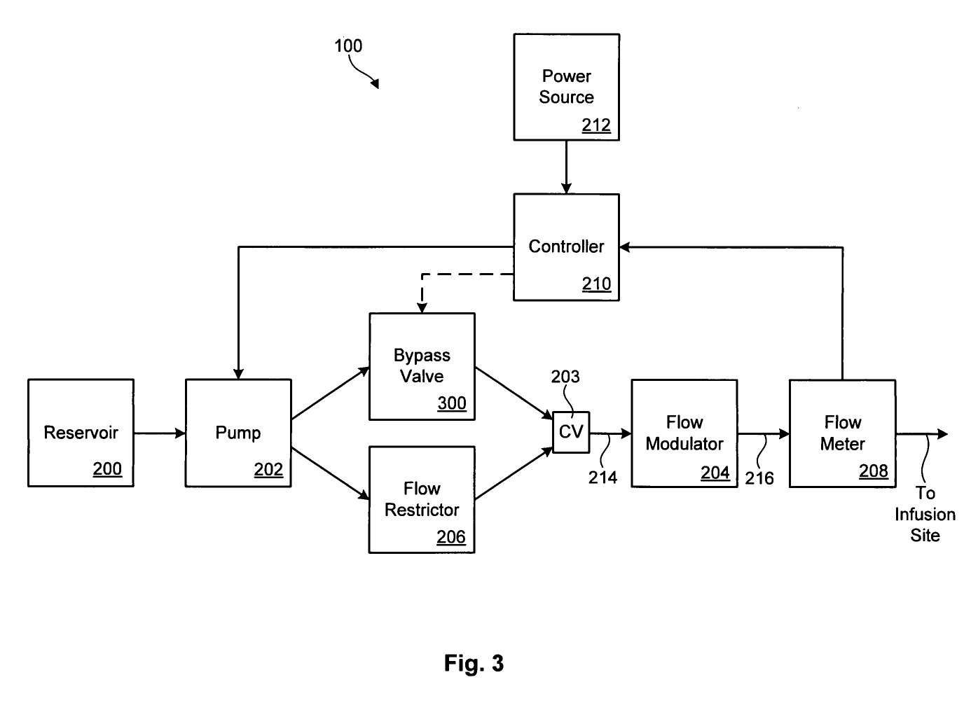

Referring to FIG. 3, in selected embodiments, a bypass valve 300 may be added to the fluid delivery device 100 to enable bolus infusions. For example, a bypass valve 300 may be provided in parallel with the flow restrictor 206. To deliver medication at basal delivery rates, the bypass valve 300 may be closed such that the flow restrictor 206 regulates the flow of liquid though the device 100. When a bolus infusion is desired, the fluid pressure may be increased (e.g., by increasing the pump speed) until it opens the bypass valve 300. This will allow additional fluid to bypass a flow restrictor 206, providing an increased delivery rate.

Similarly, when the bolus has terminated, the liquid pressure may be decreased (e.g., by decreasing the pump speed) until the bypass valve 300 closes. This will terminate the flow of additional liquid around the flow restrictor 206, thereby returning to the basal delivery rate. In selected embodiments, the bypass valve 300 is a passive valve, meaning that only an increase or decrease in pressure is required to open or close the valve 300. In other embodiments, the bypass valve 300 is an active device controlled by the controller 210. For example, the controller 210 may increase the pump speed and simultaneously open the bypass valve 300 to deliver a bolus.

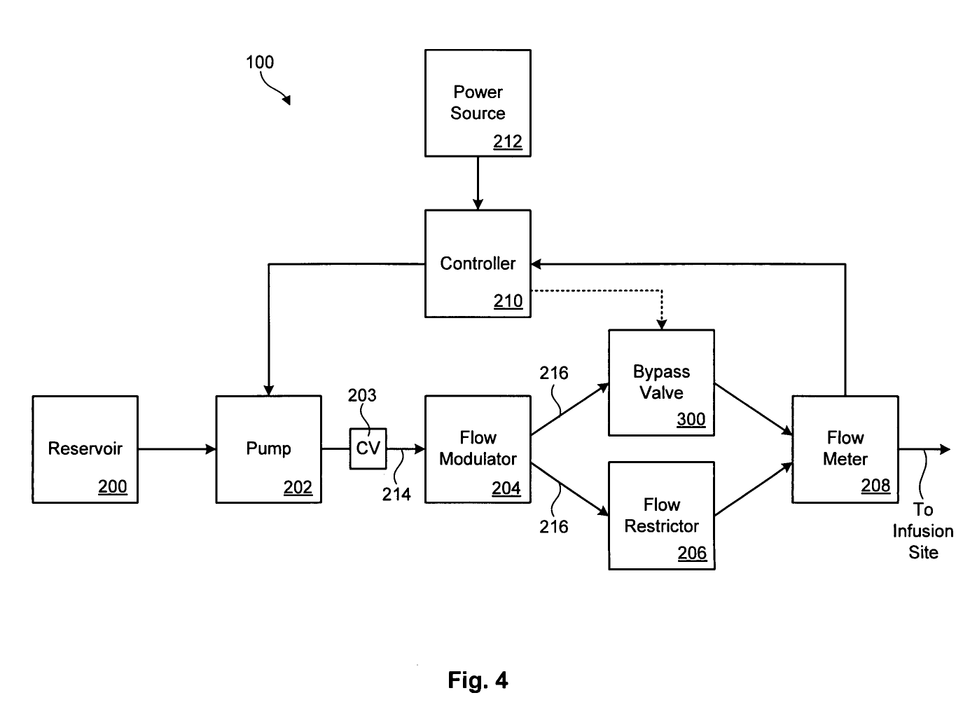

Like the previous example, the fluid delivery device 100 illustrated in FIG. 3 is provided only by way of example and is not intended to be limiting. Various components may be re-arranged or possibly added or removed without altering the function or principles of operation of the device 100. For example, in the embodiment of FIG. 3, the flow meter 208 may act as a flow restrictor 206 to create back pressure sufficient to expand the volume in the volume expansion zone within the flow modulator 204 when the second flow rate is lower than the first flow rate. Thus, in some embodiments, the flow meter 208 can be the flow restrictor 206. Referring to FIG. 4, in selected embodiments, the bypass valve 300 and flow restrictor 206 are placed downstream from the flow modulator 204. Thus, the present invention may be embodied in other forms without departing from its basic principles or essential characteristics.

Referring to FIG. 5A, as previously explained, a flow modulator 204 in accordance with the invention may include a volume expansion zone 500 as well as a mechanism 502 to act on the boundaries of the volume expansion zone 500 to compress liquid therein. For example, in selected embodiments, the volume expansion zone 500 may be provided in the form of an expandable bag 504 or bellows 504. This bag 504 or bellows 504 may expand as fluid enters the expansion zone 500 and contract as fluid exits the expansion zone 500. A spring 502 or elastomeric member 502 may act on the boundaries of the volume expansion zone 500 to compress the liquid therein. The volume expansion zone 500 combined with the spring 502 or elastomeric member 502 may smooth out irregularities in the flow rate of a first fluid stream 214, thereby generating a second fluid stream 216 with a substantially more uniform flow rate.

Referring to FIG. 5B, in another embodiment, the volume expansion zone 500 may be implemented with a flexible diaphragm 506. The flexible diaphragm 506 may expand as fluid enters the expansion zone 500 and contract as fluid exits the expansion zone 500. In certain embodiments, the diaphragm 506 may be made of an elastomeric material that will act on the boundaries of the volume expansion zone 500 to compress the liquid therein. In other embodiments, a spring or elastomeric material (not shown) may act on the diaphragm 506 to compress the liquid in the expansion zone 500.

The flow modulators 204 described in FIGS. 5A and 5B are provided only by way of example and are not intended to be limiting. Indeed, various different types of flow modulators not disclosed herein may be used with the present invention. For example, the flow modulator 204 may be as simple as an elastomeric bag, a piece of tubing with elastic walls, or a spring acting on a piston. The flow modulator 204 could also contain a compressible fluid or gas, contained within a leak-tight enclosure, which is used to contract the expansion zone 500. Another embodiment of a flow modulator 204 may include foam or sponge-like material that compresses as the expansion zone expands under pressure, while contracting the expansion zone when the pressure decreases. These represent just a few examples of possible flow modulators 204.

It will be appreciated by reference to the FIGS. 1-5B that in accordance with at least one embodiment of the present invention, a device to deliver fluid to a target site at a substantially uniform delivery rate may include means for generating a first fluid stream having a first flow rate. These means may include without limitation piezo pumps, diaphragm-type pumps, centrifugal pumps, peristaltic pumps, piston-type pumps, and the like. These means may also include pressure differentials of a type known in the art to induce fluid flow, reservoirs and other fluid containers or conduits under the force of gravity, other ways know in the art, and combinations thereof.

The device may further include means for smoothing out irregularities in the first flow rate to thereby generate a second fluid stream. The second fluid stream may have a second flow rate that is substantially more uniform than the first flow rate. These means may include the volume expansion zone and flow modulator 204 and its components as discussed herein.

The device may further include means for measuring the second flow rate. These means may include without limitation thermal differentials, pressure differentials across an orifice, pressure differentials across a petot tube, pressure differentials across a venturi, other ways known to those of skill in the art, and combinations thereof. The device may also include means for comparing the second flow rate to a target flow rate, and means for substantially aligning the second flow rate with the target flow rate. These means may include a controller, software, firmware, hardware, adjusting the pump speed, including the pump amplitude, pump duty cycle, pump frequencies, and the like, alone or in combination. In certain embodiments, the device may also include means for restricting a flow of the first or second fluid stream. These means may include those discussed in combination with the flow restrictor 206. In certain embodiments, the device may enable the first or second fluid stream to bypass a means for restricting the flow by means including spring-loaded check valves, solenoid valves, manual valves, pluming configurations, other bypass ways known in the art, and combinations thereof.

In some embodiments, as discussed above, it is understood that the flow meter 208, may serve as a means of creating backpressure downstream of the flow modulator 204 that serves to cause the volume in the flow modulator 204 to expand when the flow rate of the second stream is lower than the first stream. This volume may be the volume of volume expansion zone.

A device according to the present invention may have more than one flow restrictor 206. For example a flow restrictor 206 may reside downstream of the flow modulator 204 for the purpose as described herein, and a second flow restrictor 206 may reside upstream of the flow modulator 204 to serve the purpose described herein, as depicted in FIG. 3.

Referring again to FIG. 1, if a liquid reservoir 200 is located a standing person's waist level 100 and the liquid entry point into the body is a knee level 104 there is a driving force for liquid to flow due to the force of gravity, even when the pump is off. Assuming there are no closed shut off valves between the liquid reservoir and the liquid entry point, there will be a tendency for liquid to flow at a rate according to the overall flow restrictions in the line. For safety purposes it may be desirable to have a flow restrictor sized such that the maximum flow possible will be at some safe, convenient rate when the pump is off. For example, in the case of local anesthetic drugs, the flow restrictor may be sized such that the maximum flow rate with approximately 2 feet head pressure is 3 cubic centimeters per hour which is also the minimum target basal rate. To achieve a target basal rate of 3-10 cubic centimeters per hour, the flow meter will detect that the flow rate needs to be increased and the controller will activate the pump. If the same device is then used for a similar application, but in this case the liquid reservoir is located a standing person's waist level, 100 and the liquid entry point into the body is at neck or shoulder level, 102, there is a gravitational force that must be overcome for liquid to flow, in addition to the flow restrictions in the line. For many pumps, at basal rates between 3-10 cubic centimeters per hour, achieving the target rate against the gravitational head and losses due to the flow restrictor is easily attained, but when a bolus rate of say 50 cubic centimeters per hour is desired, there is too much resistance to flow and the pump cannot achieve the desired rate. In this case, a bypass valve, 300, is desirable. The bypass (see FIGS. 3 and 4) may be a spring loaded check valve that opens when the line pressure reaches 2 psi which is equivalent to approximately 4.6 feet of water head due to gravity. In most situations where the basal rates are targeted, the bypass valve would remain closed. In the case described above where the pump is off and there is approximately 2 feet of head due to gravity, the bypass valve would remain closed. But when the pump rate is increased to achieve a bolus rate, the bypass valve would open, enabling the bolus rate to be achieved, even with the flow restrictor 206 in place. The bypass valve also could be actively opened by the controller, 210, when the flow meter detects that the desired flow rate is not being attained.

Variations of the piping scheme described above may be used to achieve similar results.

An exemplarily device may consist of the following: a variable volume liquid reservoir containing the drugs ropivacaine or bupivacaine or the like, a piezo electric pump with passive check-valves, a line that flows to flow restrictor consisting of a length of small diameter tubing sized such that the flow of the drug with 2 feet head pressure is 3 cc per hour and a spring loaded bypass check valve that opens when the pressure in the line exceeds 4.6 feet of head pressure (2 psi). The bypass check valve and the flow restrictor flow to an elastomeric balloon with an inlet and an outlet. This balloon serves as the flow modulator. Following the balloon, the flow runs to a differential temperature type flow meter. There is sufficient resistance to flow from the flow meter to create a backpressure when the piezo pump strokes resulting in a partial filling of the balloon during the stroke. Between pump strokes, the balloon walls compress on the liquid contents resulting in flow through the flow meter since the check valve on the piezo pump prevents back flow to the reservoir. Following the flow meter the flow is to an entry point of the body to a catheter that has been placed near nerves which when bathed in the drug will prevent pain from a surgery. The flow meter provides flow rate data to a controller that will increase the piezo pump duty cycle to achieve a desired basal flow rate that typically is between 3-10 cc/hour. If the patient begins to feel considerable pain because the basal rate is too low, the patient may activate a bolus where the pump rate increases to 50 cc/h for 15 minutes. During the bolus, the bypass valve may open. After 15 minutes, the controller will reduce the pump rate to resume the basal rate. The controller may have been programmed to prevent another bolus from occurring for a set period of time, say 8 hours.

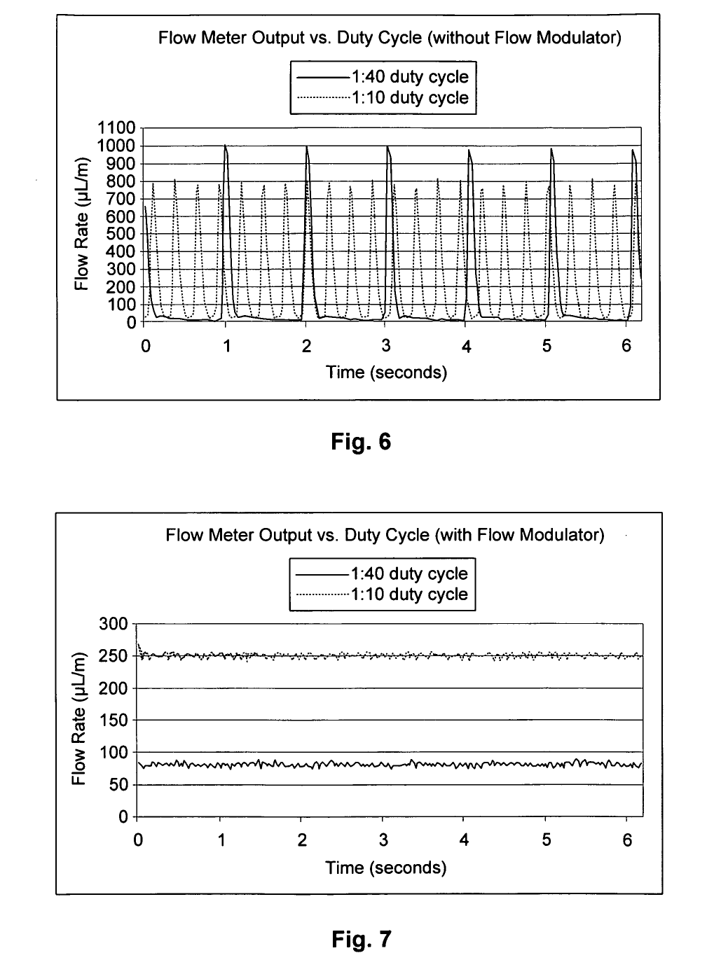

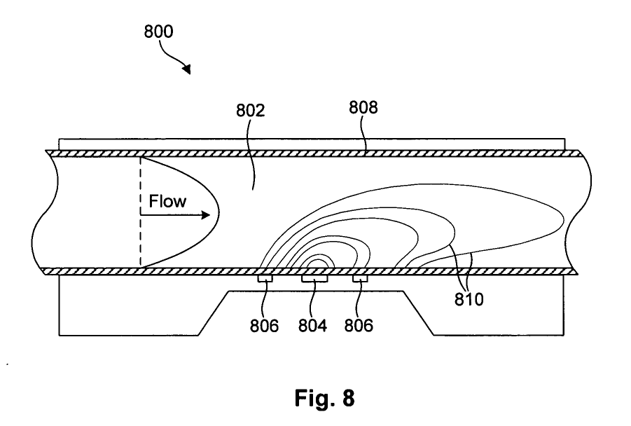

Referring to FIG. 6, a graph is illustrated that shows the results of an experimental fluid delivery device without the flow modulator 204 described herein. In the experiment, water was pumped from first beaker using a piezo pump to another beaker placed on a precision weight balance. The pump was operated at 40 Hertz with a 1:40 duty cycle such that the pump actuated approximately once per second. The pump was then operated at a 1:10 duty cycle such that the pump actuated about 3.7 times per second. The flow rate was measured with a thermal/temperature type measurement device similar to the device described in FIG. 8. The flow rate was also measured gravimetrically by examining the slope of a weight versus time curve. The water was pumped through approximately nine feet of 1/32 inch ID polymer tubing. As can be seen from the graph, the flow rates generated numerous sharp peaks, showing the irregular (i.e, pulsatile) nature of the flow rates.

Referring to FIG. 7, a graph is illustrated that shows the results of an experimental fluid delivery device with the flow modulator 204 described herein. In this experiment, a polyurethane balloon with an inlet and outlet was placed in the fluid path between the pump and the flow meter. The balloon size was approximately one inch long with a diameter of approximately one inch and with a conical transition from the inlet and to the outlet such that the overall length of the balloon was approximately two inches. Water was then pumped through the device and the flow rate measured as previously described in association with FIG. 6. As can be seen from the graph, the elastic walls of the balloon, the flow resistance provided by the tubing downstream from the balloon, and a check valve on the pump provided significant flow smoothing compared to the experiment of FIG. 6.

Referring to FIG. 8, in selected embodiments, a flow meter 800 for use with the invention may be used to calculate a temperature differential in order to determine a flow rate. For example, in selected embodiments, the flow meter 800 may include a heat source 804 and two temperature sensors 806 located on either side of the heat source 804. The heat source 804 and temperature sensors 806 may be placed on an exterior side of a section of tubing 808 such that they are in thermal contact therewith. A liquid may then be passed through the tubing 808. The heat source 804 slightly heats the liquid as it passes by the heat source 804 (as shown by the temperature gradient lines 810).

A temperature differential may then be calculated between the temperature sensors 806. As the flow rate increases, the temperature differential measured at the sensors 806 may decrease. Similarly, as the flow rate decreases the temperature differential measured at the sensors 806 may increase. A flow meter 800 like that illustrated in FIG. 8 has been shown to generate flow measurements with a high degree of accuracy and sensitivity. Nevertheless, the invention is not limited to the illustrated flow meter 800, but may include other types of flow meters not disclosed herein.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

What is claimed is:

1. a device to deliver a fluid to a target site at a substantially uniform delivery: rate, the device comprising:
a pump to generate a first fluid stream having a first flow rate, the pump having a pump speed;
a flow modulator receiving said first fluid stream, said flow modulator having an opening to permit the exit of a second fluid stream having a second flow rate; and
wherein the flow modulator accumulates fluid when the first flow rate is greater than the second flow rate and expels fluid when the second flow rate is greater than the first flow rate.

2. The device of claim 1, further comprising a flow meter to measure the second flow rate.

3. The device of claim 1, further comprising a controller configured to compare the second flow rate to a target flow rate and adjust the pump speed to substantially align the second flow rate with the target flow rate.

4. The device of claim 1, further comprising a check valve to prevent fluid backflow from the flow modulator to the pump.

5. The device of claim 1, further comprising a flow restrictor to regulate one of the first fluid stream and the second fluid stream.

6. The device of claim 1, wherein the pump operates intermittently such that the first flow rate is substantially pulsatile.

7. The device of claim 1, wherein the pump is selected from the group consisting of a piezo pump, a diaphragm-type pump, a centrifugal pump, a peristaltic pump, and a piston-type pump.

8. The device of claim 1, wherein the flow modulator comprises a volume expansion zone having a volume that expands to accumulate fluid when the first flow rate is greater than the second flow rate.

9. The device of claim 8, wherein the volume expansion zone contracts to expel fluid when the first flow rate is less than the second flow rate.

10. The device of claim 8, wherein the volume expansion zone comprises a bellows in communication with a biasing member to allow the volume to expand and contract.

11. The device of claim 8, wherein the volume expansion zone comprises an expandable diaphragm to allow the volume to expand and contract.

12. The device of claim 8, wherein the volume expansion zone comprises an elastomeric balloon to allow the volume to expand and contract.

13. The device of claim 1, further comprising a fluid reservoir in communication with the pump to retain the fluid prior to delivery to the target site.

14. The device of claim 3, further comprising a power source for supplying power to the controller.

15. The device of claim 5, further comprising a bypass valve to enable one of the first fluid stream and the second fluid stream to bypass a flow restrictor.

16. A method to deliver a fluid to a target site at a substantially uniform delivery rate, the method comprising:
generating a first fluid stream having a first flow rate;
introducing the first fluid stream into a flow modulator;
permitting a second fluid stream having a second flow rate that is substantially more uniform than the first flow rate to exit the flow modulator.

17. The method of claim 16, further comprising measuring the second flow rate.

18. The method of claim 17, further comprising comparing the second flow rate to a target flow rate.

19. The method of claim 18, further comprising adjusting the first flow rate to substantially align the second flow rate with the target flow rate.

20. The method of claim 16, further comprising preventing the first fluid stream from substantially reversing a direction of flow.

21. The method of claim 16, further comprising providing a flow restrictor to regulate a flow of one of the first fluid stream and the second fluid stream.

22. The method of claim 21, further comprising providing a bypass valve to enable one of the first fluid stream and the second fluid stream to bypass a flow restrictor.

23. The method of claim 16, wherein the first fluid stream is substantially pulsatile.

24. The method of claim 16, wherein introducing the first fluid stream into a flow modulator further comprises receiving the first fluid stream into a volume expansion zone.

25. The method of claim 24, further comprising expanding a volume in the volume expansion zone to accumulate fluid within the flow modulator when the first flow rate is greater than the second flow rate.

26. The method of claim 24, further comprising contracting a volume in the volume expansion zone to expel fluid from the flow modulator when the first flow rate is less than the second flow rate.

27. A device to deliver a fluid to a target site at a substantially uniform delivery rate, the device comprising:
means for generating a first fluid stream having a first flow rate;
means for smoothing out irregularities in the first flow rate, and thereby generate a second fluid stream having a second flow rate substantially more uniform than the first flow rate;
means for measuring the second flow rate;
means for comparing the second flow rate to a target flow rate; and
means for substantially aligning the second flow rate with the target flow rate.

28. The device of claim 18, further comprising means for restricting a flow of one of the first fluid stream and the second fluid stream.

29. The device of claim 19, further comprising means for enabling one of the first fluid stream and the second fluid stream to bypass a means for restricting the flow.

30. A device to deliver a fluid to a target site at a substantially uniform delivery rate, the device comprising:
a pump to generate a first fluid stream having a first flow rate, the pump having a pump speed;
a volume expansion zone to receive the first fluid stream and to exit a second fluid stream having a second flow rate, said volume expansion zone having a volume that expands to accumulate fluid when the first flow rate is greater than the second flow rate and that contracts to expel fluid when the first flow rate is less than the second flow rate;
a valve positioned between the pump and the volume expansion zone to prevent fluid back flow from the volume expansion zone to the pump;
a flow restrictor to facilitate fluid accumulation in the volume expansion zone;
a flow meter to measure a second flow rate exiting the volume expansion zone; and
a controller configured to compare the second flow rate to a target flow rate and adjust the pump speed to substantially align the second flow rate with the target flow rate.

31. A device to deliver a fluid to a target site at a substantially uniform delivery rate, the device comprising:
a pump to generate a first fluid stream having a first flow rate, the pump having a pump speed;
a volume expansion zone comprising a flexible diaphragm, expandable when the first flow rate is greater than the second flow rate to accumulate fluid within the volume expansion zone, and contractable when the first flow rate is less than the second flow rate to urge fluid out of the volume expansion zone;
a valve positioned between the pump and the volume expansion zone to prevent fluid backflow from the volume expansion zone to the pump;
a flow restrictor to facilitate fluid accumulation in the volume expansion zone;
a flow meter to measure a second flow rate of a second fluid stream exiting the volume expansion zone;
a controller configured to compare the second flow rate to a target flow rate and adjust the pump speed to substantially align the second flow rate with the target flow rate; and;
a bypass valve to enable one of the first fluid stream and the second fluid stream to bypass a flow restrictor.
